SEQUENCE LISTING (81 sequences)

SEQ ID NO: 1 - DNA, 22 bp, Artificial
  Synthetic Oligonucleotide Primer
acctccaatcactcaccaacct

SEQ ID NO: 2 - DNA, 22 bp, Artificial
  Synthetic Oligonucleotide Primer
gaaagccctacgaaccactgaa

SEQ ID NO: 3 - DNA, 22 bp, Artificial
  Synthetic Oligonucleotide Primer
cgctctagccatggcgttagta

SEQ ID NO: 4 - DNA, 22 bp, Artificial
  Synthetic Oligonucleotide Primer
cctatcaggcagtaccacaagg

SEQ ID NO: 5 - DNA, 26 bp, Artificial
  Synthetic Oligonucleotide Primer
atacccatgttywcagcattatcaga

SEQ ID NO: 6 - DNA, 24 bp, Artificial
  Synthetic Oligonucleotide Primer
acccatgttywcagcattatcaga

SEQ ID NO: 7 - DNA, 25 bp, Artificial
  Synthetic Oligonucleotide Primer
atacccatgttywcagcattatcag

SEQ ID NO: 8 - DNA, 22 bp, Artificial
  Synthetic Oligonucleotide Primer
acccatgttywcagcattatca

SEQ ID NO: 9 - DNA, 26 bp, Artificial
  Synthetic Oligonucleotide Primer
ctatttgttcytgaagggtactagta

SEQ ID NO: 10 - DNA, 25 bp, Artificial
  Synthetic Oligonucleotide Primer
ctatttgttcytgaagggtactagt

SEQ ID NO: 11 - DNA, 24 bp, Artificial
  Synthetic Oligonucleotide Primer
atttgttcytgaagggtactagta

SEQ ID NO: 12 - DNA, 22 bp, Artificial
  Synthetic Oligonucleotide Primer
atttgttcytgaagggtactag

SEQ ID NO: 13 - DNA, 26 bp, Artificial
  Synthetic Oligonucleotide Primer
attcctatgttyatggcattrtcaga

SEQ ID NO: 14 - DNA, 24 bp, Artificial
  Synthetic Oligonucleotide Primer
ttcctatgttyatggcattrtcag

SEQ ID NO: 15 - DNA, 25 bp, Artificial
  Synthetic Oligonucleotide Primer
ttcctatgttyatggcattrtcaga

SEQ ID NO: 16 - DNA, 23 bp, Artificial
  Synthetic Oligonucleotide Primer
tcctatgttyatggcattrtcag

SEQ ID NO: 17 - DNA, 26 bp, Artificial
  Synthetic Oligonucleotide Primer
kaatttgctcttgctgkgtgctagtt

SEQ ID NO: 18 - DNA, 25 bp, Artificial
  Synthetic Oligonucleotide Primer
aatttgctcttgctgkgtgctagtt

SEQ ID NO: 19 - DNA, 25 bp, Artificial
  Synthetic Oligonucleotide Primer
kaatttgctcttgctgkgtgctagt

SEQ ID NO: 20 - DNA, 23 bp, Artificial
  Synthetic Oligonucleotide Primer
kaatttgctcttgctgkgtgcta

SEQ ID NO: 21 - DNA, 30 bp, Artificial
  Synthetic Oligonucleotide Primer
actagtaaactgagccaggagaaacggact

SEQ ID NO: 22 - DNA, 29 bp, Artificial
  Synthetic Oligonucleotide Primer
ctagtaaactgagccaggagaaacggact

SEQ ID NO: 23 - DNA, 29 bp, Artificial
  Synthetic Oligonucleotide Primer
actagtaaactgagccaggagaaacggac

SEQ ID NO: 24 - DNA, 28 bp, Artificial
  Synthetic Oligonucleotide Primer
ctagtaaactgagccaggagaaacggac

SEQ ID NO: 25 - DNA, 24 bp, Artificial
  Synthetic Oligonucleotide Primer
ctagccgagtagygttgggtygcg

SEQ ID NO: 26 - DNA, 23 bp, Artificial
  Synthetic Oligonucleotide Primer
tagccgagtagygttgggtygcg

SEQ ID NO: 27 - DNA, 23 bp, Artificial
  Synthetic Oligonucleotide Primer
ctagccgagtagygttgggtygc

SEQ ID NO: 28 - DNA, 21 bp, Artificial
  Synthetic Oligonucleotide Primer
tagccgagtagygttgggtyg

SEQ ID NO: 29 - DNA, 26 bp, Artificial
  Synthetic Oligonucleotide Primer
aatgaggaagctgcagaatgggayag

SEQ ID NO: 30 - DNA, 25 bp, Artificial
  Synthetic Oligonucleotide Primer
atgaggaagctgcagaatgggayag

SEQ ID NO: 31 - DNA, 25 bp, Artificial
  Synthetic Oligonucleotide Primer
aatgaggaagctgcagaatgggaya

SEQ ID NO: 32 - DNA, 23 bp, Artificial
  Synthetic Oligonucleotide Primer
tgaggaagctgcagaatgggaya

SEQ ID NO: 33 - DNA, 25 bp, Artificial
  Synthetic Oligonucleotide Primer
aaggaagtaatcaatgaggaagcag

SEQ ID NO: 34 - DNA, 24 bp, Artificial
  Synthetic Oligonucleotide Primer
aggaagtaatcaatgaggaagcag

SEQ ID NO: 35 - DNA, 24 bp, Artificial
  Synthetic Oligonucleotide Primer
aaggaagtaatcaatgaggaagca

SEQ ID NO: 36 - DNA, 22 bp, Artificial
  Synthetic Oligonucleotide Primer
aggaagtaatcaatgaggaagc

SEQ ID NO: 37 - DNA, 26 bp, Artificial
  Synthetic Oligonucleotide Primer
kaatttgctcttgctgkgtgctagtt

SEQ ID NO: 38 - DNA, 22 bp, Artificial
  Synthetic Oligonucleotide Primer
gcgtctagccatggcgttagta

SEQ ID NO: 39 - DNA, 30 bp, Artificial
  Synthetic Oligonucleotide Primer
atgtagaggggctgttgaaaaaaccctggt

SEQ ID NO: 40 - DNA, 25 bp, Artificial
  Synthetic Oligonucleotide Primer
tttaaatatgatgctaaacatagtg

SEQ ID NO: 41 - DNA, 25 bp, Artificial
  Synthetic Oligonucleotide Primer
ctgcagaatgggatagattgcatcc

SEQ ID NO: 42 - DNA, 25 bp, Artificial
  Synthetic Oligonucleotide Primer
tttaaacaccatgttaaatacagtg

SEQ ID NO: 43 - DNA, 25 bp, Artificial
  Synthetic Oligonucleotide Primer
ggataggctacatccagtgcatgca

SEQ ID NO: 44 - DNA, 28 bp, Artificial
  Synthetic Oligonucleotide Primer
attacatccagtgcaggcagggcctatc

SEQ ID NO: 45 - DNA, 28 bp, Artificial
  Synthetic Oligonucleotide Primer
agcagctatgcaaatgctaaaggatact

SEQ ID NO: 46 - DNA, 25 bp, Artificial
  Synthetic Oligonucleotide Primer
ataggstacatccactgcaggcagg

SEQ ID NO: 47 - DNA, 25 bp, Artificial
  Synthetic Oligonucleotide Primer
traatgccataggrggacaycaagg

SEQ ID NO: 48 - DNA, 21 bp, Artificial
  Synthetic Oligonucleotide Primer
tctggggcatyarrcarctcc

SEQ ID NO: 49 - DNA, 20 bp, Artificial
  Synthetic Oligonucleotide Primer
ggtkrrtatccctgcctaay

SEQ ID NO: 50 - DNA, 21 bp, Artificial
  Synthetic Oligonucleotide Primer
tgctctggaarrcwcatytgc

SEQ ID NO: 51 - DNA, 22 bp, Artificial
  Synthetic Oligonucleotide Primer
ggtatatwaramtattyataat

SEQ ID NO: 52 - DNA, 22 bp, Artificial
  Synthetic Oligonucleotide Primer
taaragaaraggggggattggg

SEQ ID NO: 53 - DNA, 20 bp, Artificial
  Synthetic Oligonucleotide Primer
aatgtgccctggaactctag

SEQ ID NO: 54 - DNA, 18 bp, Artificial
  Synthetic Oligonucleotide Primer
rctgtgccttggaatrct

SEQ ID NO: 55 - DNA, 20 bp, Artificial
  Synthetic Oligonucleotide Primer
gctgtgccttggaactccac

SEQ ID NO: 56 - DNA, 20 bp, Artificial
  Synthetic Oligonucleotide Primer
aacatgacctggctgcaatg

SEQ ID NO: 57 - DNA, 20 bp, Artificial
  Synthetic Oligonucleotide Primer
aacatgacctggatgsagtg

SEQ ID NO: 58 - DNA, 20 bp, Artificial
  Synthetic Oligonucleotide Primer
aacatgacatggatagaatg

SEQ ID NO: 59 - DNA, 27 bp, Artificial
  Synthetic Oligonucleotide Primer
ttrttggcattggacaartgggcaart

SEQ ID NO: 60 - DNA, 23 bp, Artificial
  Synthetic Oligonucleotide Primer
ggaattrgataartgggcaagtt

SEQ ID NO: 61 - DNA, 25 bp, Artificial
  Synthetic Oligonucleotide Primer
aargatttattagcattggacartt

SEQ ID NO: 62 - DNA, 27 bp, Artificial
  Synthetic Oligonucleotide Primer
tattgsaattggacaartgggcaagtt

SEQ ID NO: 63 - DNA, 31 bp, Artificial
  Synthetic Oligonucleotide Primer
gatttgttagaattggataaatgggcaagtc

SEQ ID NO: 64 - DNA, 26 bp, Artificial
  Synthetic Oligonucleotide Primer
agtttttgctgtgctttctataataa

SEQ ID NO: 65 - DNA, 24 bp, Artificial
  Synthetic Oligonucleotide Primer
atttttgctgtactytctatagtr

SEQ ID NO: 66 - DNA, 27 bp, Artificial
  Synthetic Oligonucleotide Primer
cgcgagcaccactamtgtgccctcgcg

SEQ ID NO: 67 - DNA, 28 bp, Artificial
  Synthetic Oligonucleotide Primer
cgcgagcttggaatrctagtggctcgcg

SEQ ID NO: 68 - DNA, 29 bp, Artificial
  Synthetic Oligonucleotide Primer
cgcgagccctggaactccacttgctcgcg

SEQ ID NO: 69 - DNA, 27 bp, Artificial
  Synthetic Oligonucleotide Primer
cgcgagctggctgcaatgggactcgcg

SEQ ID NO: 70 - DNA, 27 bp, Artificial
  Synthetic Oligonucleotide Primer
cgcgagctggatgsagtgggactcgcg

SEQ ID NO: 71 - DNA, 28 bp, Artificial
  Synthetic Oligonucleotide Primer
cgcgagatggatagaatggacactcgcg

SEQ ID NO: 72 - DNA, 27 bp, Artificial
  Synthetic Oligonucleotide Primer
cgcgagtggcattggacaagtctcgcg

SEQ ID NO: 73 - DNA, 28 bp, Artificial
  Synthetic Oligonucleotide Primer
cgcgaggatttattagcattggctcgcg

SEQ ID NO: 74 - DNA, 29 bp, Artificial
  Synthetic Oligonucleotide Primer
cgcgagaagaattattggaattgctcgcg

SEQ ID NO: 75 - DNA, 29 bp, Artificial
  Synthetic Oligonucleotide Primer
cgcgaggacaaatgggcaagtttctcgcg

SEQ ID NO: 76 - DNA, 29 bp, Artificial
  Synthetic Oligonucleotide Primer
cgcgaggatttgttagaattggactcgcg

SEQ ID NO: 77 - DNA, 28 bp, Artificial
  Synthetic Oligonucleotide Primer
cgcgagggataaatgggcaagtctcgcg

SEQ ID NO: 78 - DNA, 27 bp, Artificial
  Synthetic Oligonucleotide Primer
ccctgtgaggaactwctgtcttcacgc

SEQ ID NO: 79 - DNA, 26 bp, Artificial
  Synthetic Oligonucleotide Primer
tctagccatggcgttagtaygagtgt

SEQ ID NO: 80 - DNA, 385 bp, Artificial
  Synthetic Oligonucleotide Primer
gaaagccctacgaaccactgaaagtccgagatgtagggggctgttgaaaaaaccctggtgtgggacaagatactcatctgcatccacaatgtcttccatgtcctcctcctctatcagggtgccgataaaacttggaatctgtagggctagggcaagtgcatcctttcatctccctgtataacaagatagcggggagggtcacgagccattttggagaactctgcaatcagctcacgaaacttggggcggctgtctgcatcactcatccagcatttgaccatgatcatgtacacatcaatggtacaaatgggtggctggggcaaacgctctcccttctccaagacggaggagatttcacttgcgaggttggtgagtgattggaggt

SEQ ID NO: 81 - DNA, 24 bp, Artificial
  Synthetic Oligonucleotide Primer
aatttaatacgactcactataggg

(19) United States
(12) Patent Application Publication
(10) Pub. No.: US 2004/0072148 A1
(43) Pub. Date: Apr. 15, 2004

(54) Simultaneous detection of HBV, HCV, and HIV in plasma samples using a multiplex capture assay

(76) Inventors: Jiuping Ji, Rockville, MD (US); Mark Manak, Laurel, MD (US); Irene Gonzalez, Baltimore, MD (US)

Correspondence Address: STERNE, KESSLER, GOLDSTEIN & FOX PLLC, 1100 NEW YORK AVENUE, N.W., WASHINGTON, DC, 20005, US

(21) Appl. No.: 10/407,897

(22) Filed: Apr. 7, 2003

Related U.S. Application Data
(63) Continuation of application No. 10/130,533.
Application No. 10/130,533 is a 371 of international application No. PCT/US00/31738, filed on Nov. 17, 2000.
(60) Provisional application No. 60/165,916, filed on Nov. 17, 1999.

(51) Int. Cl. C12Q 1/70; C12P 19/34

(52) U.S. Cl. 435/5; 435/91.2

(57) ABSTRACT

The present invention is directed to a capture assay to simultaneously screen for HBV, HCV and HIV nucleic acids in samples such as plasma. The nucleic acids including both viral DNA and RNA are purified from the plasma samples in a single extraction procedure. In one embodiment, a mixture of degenerate biotin-labelled PCR primers specific for the HBV, HCV, HIV-1 type M and HIV-1 type O are used to amplify any of these viruses which may be present in plasma. Amplified products are captured by hybridization to immobilized capture sequence, and thereafter detected. An internal control vector containing a synthetic fragment flanked by sequences corresponding to the HBV primers was designed to monitor sample recovery during extraction, amplification and detection. All major subtypes of HBV, HCV and HIV including HIV-1 type O have been confirmed and detected by the assay.

### BACKGROUND OF THE INVENTION

1. Field of the Invention

The present invention relates to detecting a nucleic acid sequence and, in particular, relates to an assay that can detect a plurality of nucleic acid sequences in a single test sample. More specifically, it relates to methods and reagents for the amplification and detection of nucleic acids from human immunodeficiency virus (HIV), hepatitis C virus (HCV), and hepatitis B virus (HBV) and combinations thereof.

2. Related Art

The primary risk to the safety of the blood supply in the United States is the potential transmission by transfusion of viral diseases such as hepatitis and HIV. Each year in the U.S., approximately 13 million blood donations are collected and the derived products are transfused into approximately 3.8 million patients (Kleinman, S., Transfusion 39:920-924 (1999)). Almost all cases of virus transmission by blood transfusion result from viral carrier donations prior to the appearance of detectable serological markers used to screen the blood. According to the CDC, about 250,000 Americans have been infected with HIV while new infection rate is about 35,000 per year. Annual hepatitis infection rate is even higher, about 150,000 to 300,000 new cases per year while 2.5% US population have chronic HBV or HCV infection. Despite the high sensitivity and specificity of most FDA approved serological tests on the market today, approximately 2 to 4 weeks may be required for an infected individual to mount a detectable antibody response to the virus, a period of time, known as the “window” period. In the US, the residual risk of blood borne virus transmission by blood and blood products is estimated to be 29.4 per million donations (Schreiber, G. B. et al., N. Engl. J. Med. 334:1685-1690 (1996)). Therefore transfusion-transmissible diseases continue to pose significant problems in the use of blood and blood products.

Nucleic acid based tests offer a sensitive and direct assay for the presence of infectious virus in blood samples. Recent implementation of these tests showed that an additional 42% of transfusion transmitted diseases associated with blood or blood products can be eliminated (Busch, M. P., Vox Sang 74 (Suppl. 2):147-154 (1998), Kleinman 1999).

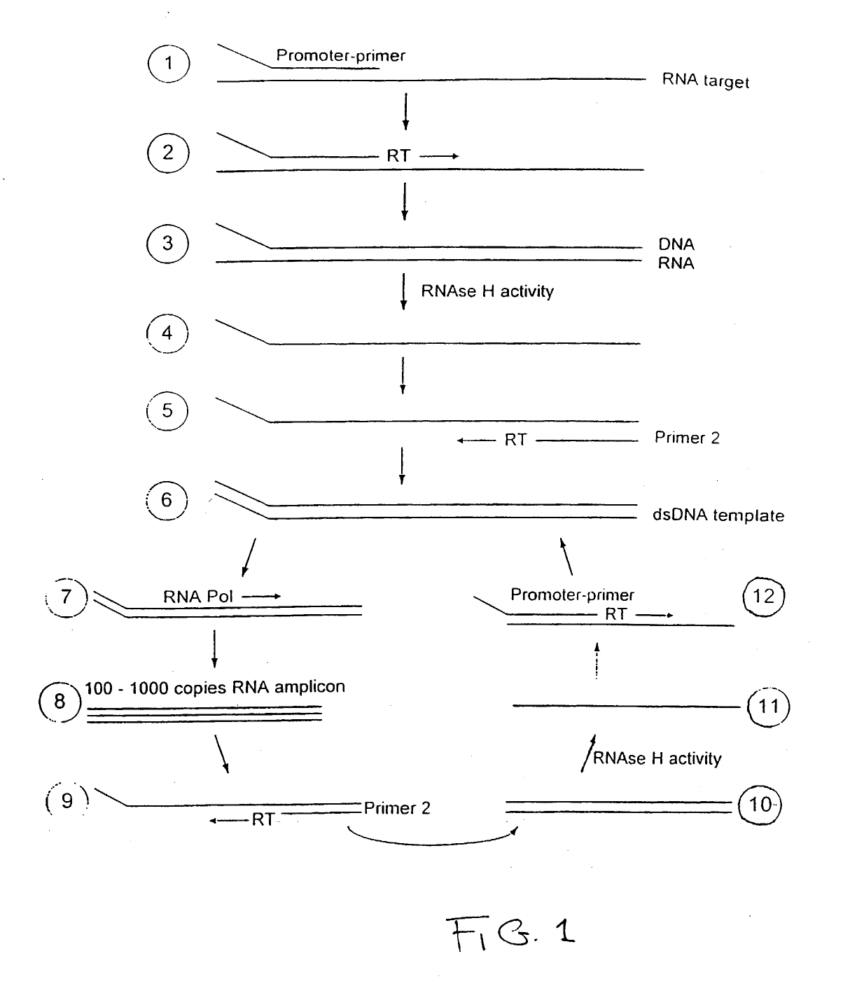

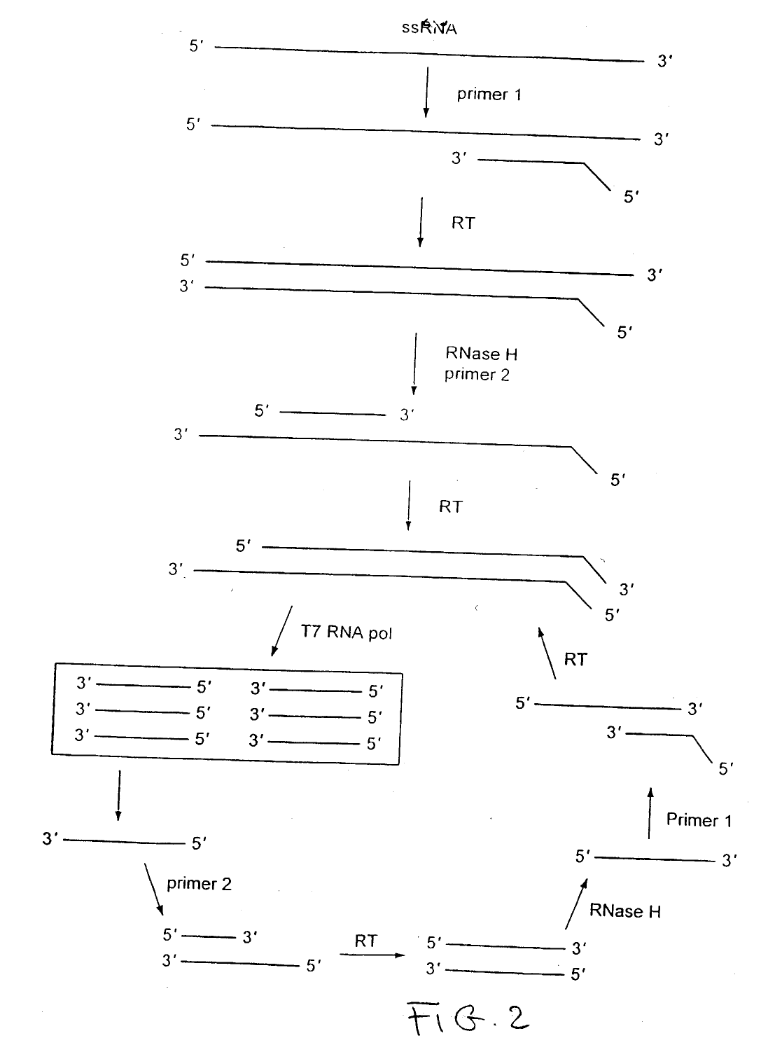

Since the advent of the polymerase chain reaction (PCR), several variations to this nucleic acid amplification reaction have been devised. Additionally, several distinct nucleic acid amplification reactions have been introduced. For example, the ligase chain reaction (LCR), transcription-mediated amplification (TMA) (see FIG. 1) and nucleic acid sequence-based amplification (NASBA)(see FIG. 2) are effective means for amplifying a nucleic acid sequence. All the above mentioned methods can be used to detect, for example, a pathogen in a test sample by amplifying a nucleic acid sequence unique to the particular pathogen (sometimes called a target sequence), then detecting the amplified nucleic acid sequences. The amplified nucleic acid sequences can be detected using techniques similar to those used in heterogeneous immunoassays.

A challenge facing the further development of amplification reactions includes the ability to reliably and quantitatively amplify and detect each target sequence in a mixed test sample containing multiple target sequences. Multiple target sequences can be detected to determine the presence of multiple pathogens that may be present in a test sample, or alternatively, multiple target sequences can be detected to quantify a target sequence present in a test sample. Unfortunately, methods for detecting multiple target sequences, for whatever purpose, are somewhat limited by the methods for detecting the signal generating groups that can be associated with the amplified sequences. In particular, in order to detect multiple target sequences, the sequences must be distinguished from one another. While such distinctions can be made by associating the sequences with different signal generating moieties, difficulties are presented when the signals from these moieties are detected. For example, when multiple fluorescent moieties are employed, each of the multiple moieties may have a distinct absorption and emission wavelength which can be employed to distinguish one sequence from another. But this detection scheme calls for a complex detection system that can excite and detect fluorophores at multiple wavelengths. Moreover, as the number of different fluorescent moieties to be detected increases, so does the complexity of the optical system employed to detect the moieties. Unfortunately, such systems are limited in the number of different sequences which can be detected because the complexity of the optical system increases in a cost prohibitive manner.

Alternatively, using multiple enzymatic signal generating moieties has been proposed to detect multiple target sequences, but such a detection scheme uses complex reagent systems to produce and inhibit signals generated by the enzymes. As a result, the predominant method for detecting multiple nucleic acid sequences is gel electrophoresis which distinguishes nucleic sequences based upon molecular weight. Gel electrophoresis, however, is a labor intensive, and therefore time consuming, method of detection which is not amenable to automation or standardization. In addition, analysis based on gel electrophoresis is not quantitative and can become complex and unreliable when more than two species of amplified nucleic acid sequences are present in a sample. Thus, there is a need for a nucleic acid amplification and detection system which is capable of detecting a plurality of target sequences in a practical manner.

### SUMMARY OF THE INVENTION

The present invention provides a multiplex capture assay to simultaneously screen for detecting the presence of HIV, HCV, HBV and combinations thereof in a sample, such as a bodily fluid or tissue. The assay comprises the steps of: (a) carrying out an amplification reaction on a sample for amplifying nucleic acids from one or more of HIV, HCV and HBV using a mixture of primers specific for HBV, HCV, HIV-1 type M and HIV-1 type O, and (b) detecting amplified products and determining whether said products are associated with HIV, HCV and HBV. A preferred detection step comprises hybridizing the amplified nucleic acids to immobilized capture sequences specific to HBV, HCV, HIV-1 type M and HIV-1 type O.

The present invention is also directed to novel primers specific to HBV, HCV, HIV-1 type M and HIV-1 type O, that can be used in multiplex amplification reactions.

The present invention is also directed to novel capture nucleic acids (probes) unique to HBV, HCV, HIV-1 type M and HIV-1 type O.

The present invention is also directed to solid supports that have been modified by adsorbing or chemically linking a probe of the present invention there to.

The present inventions is also directed to kits comprising primers and capture nucleic acids (probes) of the present invention.

BRIEF DESCRIPTION OF FIGURES

FIG. 1 is a schematic representation of MTA.

FIG. 2 is a schematic representation of NASBA.

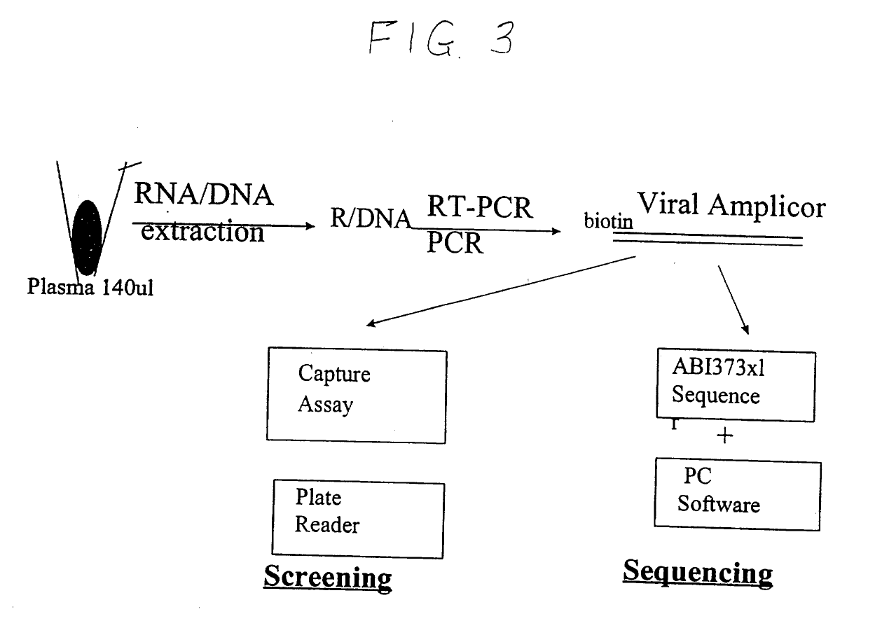

FIG. 3 is a schematic representation of a preferred embodiment of the capture assay.

### DETAILED DESCRIPTION OF PREFERRED EMBODIMENTS

The present invention provides practical methods and reagents for a rapid, specific and sensitive diagnostic assay for testing for multiple viral agents in a test sample. Samples include human bodily fluids and tissues. Useful bodily fluids include blood, saliva, semen and vaginal secretions. Useful tissues include thymus and liver. Also contemplated are blood products such as plasma, serum and white blood cells. Viruses that can be detected by the method disclosed herein include any subtypes of HCV, HBV, HIV-1-M and HIV-1-O.

According to the present invention, viral RNA or DNA can be detected without isolating the viral particles first. While nucleic acids can be first extracted from the sample, it is contemplated that amplification can take place without the extraction of nucleic acids from the sample. Most preferably, nucleic acids are extracted in a single-step extraction.

An amplification protocol is carried out by amplifying particular nucleic acid sequences using primers specific to HBV, HCV, HIV-1 type M and HIV-1 type O. Useful amplification methods include PCR, RT-PCR, TMA and NASBA. Primers are typically modified to include T7 or T3 promoter region sequences for TMA and NASBA. The primers may be used in unlabeled or labeled form. Useful labeling agents include any known nucleic acid labeling agent, including biotin, fluorophores, quenching molecules and radioactive ions. Biotin is a preferred labeling agent. Primers can range in length between about 10 bases (b) to about 500 b. More preferably, primers should range in length from about 10 b to about 100 b. Even more preferably, primers range in length from 15 b to 50 b. Most preferably, primers should range in length between about 18 b and about 40 b.

The presence of specific viral nucleic acid sequences in the sample is determined by detecting the amplified products hybridized to the capture nucleic acid sequence. Detection can be carried out by measurements of colorimetric reaction products, fluorescence, or radioactivity appropriate to the labeling reagent incorporated into the amplified products. Also, it is possible to measure a reduction in a signal from a labeling reagent incorporated into the capture nucleic acid by quenching by the amplified products substituted with an appropriate quenching reagent.

An internal control containing a synthetic fragment flanked by sequences corresponding to the HBV primers is used to monitor sample recovery during extraction, amplification and detection. An internal control is a nucleic acid sequence, unrelated to any capture nucleic acid sequence specific to a viral nucleic acid used in the assay, flanked by sequences amplifiable by the primers used in the assay.

Definitions

In order to aid in understanding the invention, several terms are defined below:

The term “primer” as used herein refers to an oligonucleotide, whether natural or synthetic, capable of acting as a point of initiation of DNA or RNA synthesis under conditions in which synthesis of a primer extension product complementary to a nucleic acid strand is induced, i.e., in the presence of four different nucleoside triphosphates and an agent for polymerization (i.e., DNA polymerase, T7 RNA polymerase or reverse transcriptase) in an appropriate buffer and at a suitable temperature. A primer is preferably a single-stranded oligodeoxyribonucleotide. The appropriate length of a primer depends on the intended use of the primer. A primer need not reflect the exact sequence of the template nucleic acid, but must be sufficiently complementary to hybridize with the template. Primers can incorporate additional features which allow for the detection or immobilization of the primer but do not alter the basic property of the primer, that of acting as a point of initiation of DNA or RNA synthesis. For example, primers may contain an additional nucleic acid sequence at the 5′ end which does not hybridize to the target nucleic acid, such as the T7 or T3 promoter region sequence to facilitate transcription. A primer may also contain an additional nucleic acid sequence at the 5′ end which does not hybridize to the target nucleic acid but which facilitates cloning of the amplified product.

The phrases “target sequence,” “target region,” and “target nucleic acid” as used herein each refer to a region of a nucleic acid which is to be amplified, detected, or otherwise analyzed.

The term “hybridization” as used herein refers the formation of a duplex structure by two single-stranded nucleic acids due to complementary base pairing. Hybridization can occur between fully complementary nucleic acid strands or between “substantially complementary” nucleic acid strands that contain minor regions of mismatch. Conditions under which only fully complementary nucleic acid strands will hybridize are referred to as “stringent hybridization conditions” or “sequence-specific hybridization conditions.” Stable duplexes of substantially complementary sequences can be achieved under less stringent hybridization conditions; the degree of mismatch tolerated can be controlled by suitable adjustment of the hybridization conditions. Those skilled in the art of nucleic acid technology can determine duplex stability empirically considering a number of variables including, for example, the length and base pair concentration of the oligonucleotides, ionic strength, and incidence of mismatched base pairs, following the guidance provided by the art (see, e.g., Sambrook et al., Molecular Cloning—A Laboratory Manual, Cold Spring Harbor Laboratory, Cold Spring Harbor, N.Y. (1989); and Wetmur, Critical Reviews in Biochem. and Mo. Biol. 26(3/4):227-259 (1991); both incorporated herein by reference).

The term “amplification” as used herein refers to any in vitro method for increasing a number of copies of a nucleotide sequence with the use of a polymerase. Nucleic acid amplification results in the incorporation of nucleotides into a DNA and/or RNA molecule or primer thereby forming a new molecule complementary to a template. The formed nucleic acid molecule and its template can be used as templates to synthesize additional nucleic acid molecules. As used herein, one amplification reaction may consist of many rounds of replication. DNA amplification reactions include, for example, polymerase chain reaction (PCR). One PCR reaction may consist of 5-100 “cycles” of denaturation and synthesis of a DNA molecule.

The phrase “capture nucleic acid sequence” or “probe” as employed herein each refer to a nucleic acid of a unique sequence capable of hybridizing to a correctly amplified fragment.

A “test sample”, as used herein, means anything suspected of containing the target sequences. The test sample can be derived from any biological source, such as a physiological fluid, including, blood, saliva, semen, ocular lens fluid, cerebral spinal fluid, sweat, urine, milk, ascites fluid, mucous, synovial fluid, peritoneal fluid, amniotic fluid, cells, and the like, or fermentation broths, cell cultures, chemical reaction mixtures and the like. Forensic materials such as, for example clothing, may also contain a target sequence and therefore are also within the meaning of the term test sample. The test sample can be used (i) directly as obtained from the source or (ii) following a pre-treatment to modify the character of the sample. Thus, the test sample can be pre-treated prior to use by, for example, preparing plasma from blood, preparing liquids from solid materials, diluting viscous fluids, filtering liquids, distilling liquids, concentrating liquids, inactivating interfering components, adding reagents, and the like. Test samples also can be pretreated to digest, restrict or render double stranded nucleic acid sequences single stranded. Moreover, test samples may be pretreated to accumulate, purify, amplify or otherwise concentrate target sequences that may be contained therein. Amplification reactions that are well known in the art can be used to amplify target sequences.

The phrase “stringent hybridization conditions,” when not specifically defined otherwise, herein refers to an overnight incubation at 42° C. in a solution comprising 50% formamide, 5×SSC (750 mM NaCl, 75 mM sodium citrate), 50 mM sodium phosphate (pH 7.6), 5× Denhardt's solution, 10% dextran sulfate, and 20 μg/ml denatured, sheared salmon sperm DNA, followed by washing the filters in 0.1×SSC at about 65° C.

Primers

5′ Primers for HBV include those polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 300 to nucleotide 400, or a portion thereof comprising at least the sequence 340 to 350. Primers can be synthesized using techniques known to those of skill in the art. Examples of useful HBV primers include polynucleotides having the sequences:

nucleotides 334-355 of A# AF214659: 5′ ACCTCCAATCACTCACCAACCT 3′

nucleotides 333-356 of A# AF214659,

nucleotides 320-360 of A# AF214659,

nucleotides 336-354 of A# AF214659, and

nucleotides 333-354 of A# AF214659.

3′ Primers for HBV include those polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 650 to nucleotide 750, or a portion thereof comprising at least the sequence 710 to 720. Primers can be synthesized using techniques known to those of skill in the art. Examples of useful 3′ HBV primers include polynucleotides having the sequences:

nucleotides 704-725 of A# AF214659; 5′ GAAAGCCCTACGAACCACTGAA3′

nucleotides 703-726 of A# AF214659;

nucleotides 705-724 of A# AF214659;

nucleotides 690-740 of A# AF214659; and

nucleotides 700-727 of A# AF214659.

5′ Primers for HCV include those polynucleotides capable of hybridizing under stringent conditions to a sequence of HCV polyprotein gene (GenBank accession number (A#) AF271632) or the complement thereof, wherein said sequence is from nucleotide 50 to nucleotide 150, or a portion thereof comprising at least the sequence 83 to 93. Primers can be synthesized using techniques known to those of skill in the art. Examples of useful 3′ HCV primers include polynucleotides having the sequences:

nucleotides 78-96 of A# AF271632, 5′ CGCTCTAGCCATGGCGTTAGTA 3′

nucleotides 79-95 of A# AF271632,

nucleotides 82-94 of A# AF271632,

nucleotides 50-100 of A# AF271632, and

nucleotides 75-95 of A# AF271632.

3′ Primers for HCV include those polynucleotides capable of hybridizing under stringent conditions to a sequence of HCV strain MD12 complete genome (GenBank accession number (A#) AF207753) or the complement thereof, wherein said sequence is from nucleotide 220 to nucleotide 320, or a portion thereof comprising at least the sequence 271 to 281. Primers can be synthesized using techniques known to those of skill in the art. Examples of useful HCV primers include polynucleotides having the sequences:

nucleotides 267-288 of A# AF207753, 5′ CCTATCAGGCAGTACCACAAGG 3′

nucleotides 266-289 of A# AF207753,

nucleotides 269-287 of A# AF207753,

nucleotides 231-297 of A# AF207753, and

nucleotides 258-300 of A# AF207753.

A number of useful 5′-HIV primers and 3′-HIV primers useful in the present invention are listed in Table 1.

In one aspect of the invention, 5′ primers for HIV-M include polynucleotides of 10 to 100 bases capable of hybridizing under stringent conditions to a nucleic acid having the sequence:

5′ ATACCCATGTT(C/T)(A/T)CAGCATTATCAGA 3′

or the complement thereof. Examples of such 5′ HIV-M primers include polynucleotides having the sequences:

5′ ACCCATGTT(C/T)(A/T)CAGCATTATCAGA 3′
5′ ATACCCATGTT(C/T)(A/T)CAGCATTATCAG 3′
5′ ACCCATGTT(C/T)(A/T)CAGCATTATCA 3′

In this aspect of the invention, useful 3′ primers for HIV-M include polynucleotides of 10 to 100 bases capable of hybridizing under stringent conditions to a nucleic acid having the sequence:

5′ CTATTTGTTC(C/T)TGAAGGGTACTAGTA 3′

or the complement thereof. Examples of such 3′ HIV-M primers include polynucleotides having the sequences:

5′ CTATTTGTTC(C/T)TGAAGGGTACTAGT 3′
5′ ATTTGTTC(C/T)TGAAGGGTACTAGTA 3′
5′ ATTGTTTC(C/T)TGAAGGGTACTAG 3′

In one aspect of the invention, 5′ primers for HIV-O include polynucleotides of 10 to 100 bases capable of hybridizing under stringent conditions to a nucleic acid having the sequence:

5′ ATTCCTATGTT(C/T)ATGGCATT(GA)TCAGA 3′

or the complement thereof. Examples of such 5′ HIV-M primers include polynucleotides having the sequences:

5′ TTCCTATGTT(C/T)ATGGCATT(GA)TCAG 3′
5′ TTCCTATGTT(C/T)ATGGCATT(GA)TCAGA 3′
5′ TCCTATGTT(C/T)ATGGCATT(GA)TCAG 3′

In this aspect of the invention, useful 3′ primers for HIV-M include polynucleotides of 10 to 100 bases capable of hybridizing under stringent conditions to a nucleic acid having the sequence:

5′ (G/T)AATTTGCTCTTGCTG(G/T)GTGCTAGTT 3′

or the complement thereof. Examples of such 3′ HIV-M primers include polynucleotides having the sequences:

5′ AATTTGCTCTTGCTG(G/T)GTGCTAGTT 3′
5′ (G/T)AATTTGCTCTTGCTG(G/T)GTGCTAGT 3′
5′ (G/T)AATTTGCTCTTGCTG(G/T)GTGCTA 3′

Capture Sequences (Probes)

The amplified products are hybridized to immobilized capture nucleic acid sequences specific to HCV, HBV, HIV-1 type M and HIV-1 type O. A capture nucleic acid sequence is a probe with a sequence unique to one of HCV, HBV, HIV-1 type M and HIV-1 type O. It is contemplated that a capture nucleic acid sequence can be the complement of a sequence unique to one of HCV, HBV, HIV-1 type M and HIV-1 type O. Useful capture nucleic acid sequences range in length from about 15 b to about 2000 b. More preferably, capture nucleic acid sequences should range in length from about 18 b to about 1000 b. More preferably, capture nucleic acid sequences range from about 18 b to about 500 b. More preferably, capture nucleic acid sequences range from about 18 b to about 100 b, and most preferably from about 20 b to about 50 b.

In one aspect of the invention probes for HBV include polynucleotides of 10 to 100 bases capable of hybridizing under stringent conditions to a nucleic acid having the sequence:

5′ ACTAGTAAACTGAGCCAGGAGAAACGGACT3′

or the complement thereof. Examples of such HBV probes include polynucleotides having the sequences:

5′CTAGTAAACTGAGCCAGGAGAAACGGACT3′
5′ACTAGTAAACTGAGCCAGGAGAAACGGAC3′
5′CTAGTAAACTGAGCCAGGAGAAACGGAC3′

In one aspect of the invention probes for HCV include polynucleotides of 10 to 100 bases capable of hybridizing under stringent conditions to a nucleic acid having the sequence:

5′ CTAGCCGAGTAG(C/T)GTTGGGT(C/T)GCG 3′

or the complement thereof. Examples of such HCV probes include polynucleotides having the sequences:

5′ TAGCCGAGTAG(C/T)GTTGGGT(C/T)GCG 3′
5′ CTAGCCGAGTAG(C/T)GTTGGGT(C/T)GC 3′
5′ TAGCCGAGTAG(C/T)GTTGGGT(C/T)G 3′

In one aspect of the invention probes for HIV-1 type M include polynucleotides of 10 to 100 bases capable of hybridizing under stringent conditions to a nucleic acid having the sequence:

5′ AAT GAG GAA GCTGCAGAATGGGAYAG 3′

or the complement thereof. Examples of such HV-1 type M probes include polynucleotides having the sequences:

5′ AT GAG GAA GCTGCAGAATGGGAYAG 3′
5′ AAT GAG GAA GCTGCAGAATGGGAYA 3′
5′ T GAG GAA GCTGCAGAATGGGAYA 3′

In one aspect of the invention probes for HIV-1 type O include polynucleotides of 10 to 100 bases capable of hybridizing under stringent conditions to a nucleic acid having the sequence:

5′ AAGGAAGTAATCAATGAGGAAGCAG 3′

or the complement thereof. Examples of such HIV-1 type O probes include polynucleotides having the sequences:

5′ AGGAAGTAATCAATGAGGAAGCAG 3′
5′ AAGGAAGTAATCAATGAGGAAGCA 3′
5′ AGGAAGTAATCAATGAGGAAGC 3′

Useful probes and primers specific for HBV, HCV or HIV are detailed in Table 1.

TABLE 1

| | | | | | | |
|---|---|---|---|---|---|---|
| TR-I | MB-ID | Primer ID | Feature | Orientation | Sequence (5′===>3′) | Bases |

Primers and probes for Multiplex

| | | | | | | |
|---|---|---|---|---|---|---|
| 4 | 46x | GAG 499-F | | forward | ATACCCATGTT(C/T)(A/T)CAGCATTATCAGA | 26 |
| 3 | 45x | GAG 710-R-BTN | 5′ biotin | reverse | 6CTATTTGTTC(C/T)TGAAGGGTACTAGTA | 27 |
| | | HIV-1-All-Cap-L | 5′-P | Probe | 5′AAT GAG GAA GCTGCAGAATGGGAYAG | 25 |
| 5 | 58x | IG-H-G-O-FWD | | forward | ATTCCTATGTT(C/T)ATGGCATT(GA)TCAGA | 26 |
| 47 | 0052xB | IG-H-G-O-REV.BTN | 5′ biotin | reverse | (G/T)AATTTGCTCTTGCTG(G/T)GTGCTAGTT | 26 |
| | | HIV-1-O-CP-L | 5′-P | Probe | AAGGAAGTAATCAATGAGGAAGCAG | 25 |
| 6 | 240x | HCV-utr-2-L | 5′ biotin | forward | GCGTCTAGCCATGGCGTTAGTA | 22 |
| 7 | 241x | HCV-utr-2-R | 5′ biotin | reverse | CCTATCAGGCAGTACCACAAGG | 22 |
| | | HCV-JJ-CAP2 | 5′-P | Probe | CTAGCCGAGTAG(C/T)GTTGGGT(C/T)GCG | 25 |
| 9 | 267 | Bio-HBV-L | | forward | ACCTCCAATCACTCACCAACCT | 22 |
| 8 | 263 | Bio-HBV-R | | reverse | GAAAGCCCTACGAACCACTGAA | 22 |
| | | CAP_HBV-301 | 5′-P | Probe | ACTAGTAAACTGAGCCAGGAGAAACGGACT | 30 |
| | | CAP_HBV-IC-519 | 5′-P | Probe | ATGTAGAGGGGCTGTTGAAAAAACCCTGGT | 30 |

Capture probes for gag region - HIV

| | | | | | | |
|---|---|---|---|---|---|---|
| 37 | 0043P | IG-G-H-A-1 | 5′-P | Probe | TTTAAATATGATGCTAAACATAGTG | 25 |
| 38 | 0044P | IG-G-H-B-2 | 5′-P | Probe | CTGCAGAATGGGATAGATTGCATCC | 25 |
| 39 | 0045P | IG-G-H-C-3 | 5′-P | Probe | TTTAAACACCATGTTAAATACAGTG | 25 |
| 40 | 0046P | IG-G-H-D-4 | 5′-P | Probe | GGATAGGCTACATCCAGTGCATGCA | 25 |
| 41 | 0047P | IG-G-H-F-5 | 5′-P | Probe | ATTACATCCAGTGCAGGCAGGGCCTATC | 28 |
| 42 | 0048P | IG-G-H-G-6 | 5′-P | Probe | AGCAGCTATGCAAATGCTAAAGGATACT | 28 |
| 43 | 0049P | JG-G-H-H-7 | 5′-P | Probe | ATAGG(G/C)TACATCCACTGCAGGCAGG | 25 |
| 44 | 0050P | IG-G-H-O-1 | 5′-P | Probe | T(A/G)AATGCCATAGG(A/G)GGACA(T/C)CAAGG | 25 |

gp41 REGION

| | | | | | | |
|---|---|---|---|---|---|---|
| 65 | 1641F | H-E-M&O-1641F | 5′-Biotin | forward | TCTGGGGCAT(C/T)A(A/G)(A/G)CA(A/G)CTCC | 21 |
| 66 | 2025R | H-E-M&O-2025R | none | reverse | GGT(G/T)(A/G)(A/G)TATCCCTGCCTAA(C/T) | 20 |
| 67 | 1696P | H-E-M&O-1696P | 5′-P | probe | TGCTCTGGAA(A/G)(A/G)C(A/T)CAT(C/T)TGC | 21 |
| 68 | 2025R | H-E-M&O-2025R | 5-P | probe | GGTATAT(A/T)A(A/G)A(A/C)TATT(C/T)ATAAT | 22 |

probes for Pol region

| | | | | | |
|---|---|---|---|---|---|
| 114 | HIV-POL-2466P | 5′-P | Probe | TAARAGAARAGGGGGGATTGGG | 22 |

probes for gp 41 region

| | | | | | |
|---|---|---|---|---|---|
| 137 | GP41-A-308P | | Probe | 5′AATGTGCCCTGGAACTCTAG | 20 |
| 138 | GP41-B-308P | | Probe | 5′(G/A)CTGTGCCTTGGAAT(G/A)CT | 18 |
| 139 | GP41-E-309P | | Probe | 5′GCTGTGCCTTGGAACTCCAC | 20 |
| 140 | GP41-A-310P | | Probe | 5′AACATGACCTGGCTGCAATG | 20 |
| 141 | GP41-B-310P | | Probe | 5′AACATGACCTGGATG(G/C)AGTG | 20 |
| 142 | GP41-E-312P | | Probe | 5′AACATGACATGGATAGAATG | 20 |
| 143 | GP41-A-318P | | Probe | 5′TT(A/G)TTGGCATTGGACAA(G/A)TGGGCAA(A/G)T | 28 |
| 144 | GP41-B-318P | | Probe | 5′GGAATT(A/G)GATAA(G/A)TGGGCAAGTT | 23 |
| 145 | GP41-C-320P | | Probe | 5′AA(A/G)GATTTATTAGCATTGGACA(G/A)TT | 25 |
| 146 | GP41-D-320P | | Probe | 5′TATTG(G/C)AATTGGACAA(G/A)TGGGCAAGTT | 27 |
| 147 | GP41-E-320 | | Probe | 5′GATTTGTTAGAATTGGATAAATGGGCAAGTC | 31 |
| 148 | GP41-A-326 | | Probe | 5′AGTTTTTGCTGTGCTTTCTATAATAA | 26 |
| 149 | GP41-C-328 | | Probe | 5′ATTTTTGCTGTACT(C/T)TCTATAGT(G/A) | 24 |
| | GP41-A-308P | 5′-P | Probe | 5′CGCGAGCACCACTA(A/C)TGTGCCCTCGCG | |
| | GP41-B-308P | 5′-P | Probe | 5′CGCGAGCTTGGAAT(G/A)CTAGTGGCTCGCG | |
| | GP41-E-309P | 5′-P | Probe | 5′CGCGAGCCCTGGAACTCCACTTGCTCGCG | |
| | GP41-A-310P | 5′-P | Probe | 5′CGCGAGCTGGCTGCAATGGGACTCGCG | |
| | GP41-B-310P | 5′-P | Probe | 5′CGCGAGCTGGATG(G/C)AGTGGGACTCGCG | |
| | GP41-E-312P | 5′-P | Probe | 5′CGCGAGATGGATAGAATGGACACTCGCG | |
| | GP41-A-318P | 5′-P | Probe | 5′CGCGAGTGGCATTGGACAAGTCTCGCG | |
| | GP41-C-320P | 5′-P | Probe | 5′CGCGAGGATTTATTAGCATTGGCTCGCG | |
| | GP41-D-320P-1 | 5′-P | Probe | 5′CGCGAGAAGAATTATTGGAATTGCTCGCG | |
| | GP41-D-320P-2 | 5′-P | Probe | 5′CGCGAGGACAAATGGGCAAGTTTCTCGCG | |
| | GP41-E-320 | 5′-P | Probe | 5′CGCGAGGATTTGTTAGAATTGGACTCGCG | |
| | GP41-E-320 | 5′-P | Probe | 5′CGCGAGGGATAAATGGGCAAGTCTCGCG | |

Extra HCV primers

| | | | | |
|---|---|---|---|---|
| 1 | HcPr98 | forward | CCCTGTGAGGAACTWCTGTCTTCACGC | 27 |
| 2 | HcPr95 | forward | TCTAGCCATGGCGTTAGTAYGAGTGT | 26 |

Capture nucleic acid sequences are immobilized onto solid support. In one embodiment, the solid support is a well or a tube associated with a microtiter plate. Solid support includes glass, plastic and agarose beads, nylon, plastic and nitrocellulose membranes, glass and plastic vials and glass and plastic tubes, and capillary tubes.

Immobilization may be carried out by any technique known to those of ordinary skill in the art.

A “hybridization platform” as used herein means a solid support material that has a defined pattern of capture probes immobilized thereon. A “solid support material” refers to any material which is insoluble, or can be made insoluble by a subsequent reaction. The solid support can be chosen for its intrinsic ability to attract and immobilize a capture probe, or the solid support can retain an additional receptor which has the ability to attract and immobilize a capture probe. The additional receptor can include a charged substance that is oppositely charged with respect to a capture probe, or the receptor molecule can be any specific binding member which is immobilized upon (attached to) the solid support material and which has the ability to immobilize the capture probe through a specific binding reaction. The receptor molecule enables the indirect binding of a capture probe to a solid support material before the performance of the assay or during the performance of the assay. The solid support material thus can be, for example, latex, plastic, derivatized plastic, magnetic or non-magnetic metal, glass or silicon surface or surfaces of test tubes, microtiter wells, sheets, beads, microparticles, chips, and other configurations known to those of ordinary skill in the art. Such materials may be used in suitable shapes, such as films, sheets, or plates, or they may be coated onto or bonded or laminated to appropriate inert carriers, such as paper, glass, plastic films, or fabrics.

Microparticles, beads and similar solid support configurations can be employed according to the present invention. These support material configurations require segregation when coated with different capture probes so that the signals associated with a given capture probe can be distinguished from a signal associated with another capture probe. Such segregation techniques are well known in the art and include fluid flow fractionation techniques which separate particulate matter based upon size.

The present invention is directed to a kit for the detection of viral agents such as HIV, HCV, HBV and combinations thereof in test samples. In one embodiment, a kit can comprise unlabeled or labeled primers specific for each of HBV, HCV, HIV-1 type M and HIV-1 type O. Useful primers for HBV, HCV and HIV include primers comprising nucleic acid sequences described above. The kit would further comprise unlabeled or labeled capture nucleic acids specific for HBV, HCV, HIV-1 type M and HIV-1 type O, immobilized on solid support. Useful probes for HBV, HCV and HIV-1 type M and HIV-1 type O include probes comprising nucleic sequences described above. Useful solid supports include wells or tubes associated with microtiter plates, nylon, plastic or nitrocellulose membranes, glass, agarose or plastic beads, glass or plastic vials or tubes, and capillary tubes. In a preferred embodiment, the capture probes would be immobilized in wells associated with a microtiter plate. The microtiter plate would be further associated with wells containing immobilized unlabeled or labeled internal control probes and with empty wells. Useful internal control probes include internal control probes comprising nucleic acid sequences described above.

The present invention is also directed to a kit comprising vials containing unlabeled or labeled primers specific for each of HBV, HCV, HIV-1 type M and HIV-1 type O and combinations thereof. Useful primers for HBV, HCV and HIV include primers comprising nucleic acid sequences described above.

The present invention is also directed to a kit comprising vials, tubes or wells containing unlabeled or labeled capture nucleic acids specific for HBV, HCV, HIV-1 type M and HIV-1 type 0. The kit may further comprise unlabeled or labeled internal control probes. In one embodiment, capture nucleic acids specific for HBV, HCV, HIV-1 type M and HIV-1 type O and internal control probes are immobilized to wells associated with a microtiter plate. In another embodiment, capture nucleic acids specific for HBV, HCV, HIV-1 type M and HIV-1 type O and internal control probes are free and not associated with solid support. In another embodiment capture nucleic acids specific for HBV, HCV, HIV-1 type M and HIV-1 type O and internal control probes are spotted onto a membrane.

In one embodiment, the capture probes can be labeled with a “signal generating system” which, as used herein, means a label or labels that generate differential signals in the presence and absence of target. Thus, a signal is generated in a “target dependent manner” which means that in the absence of target sequence, a given signal is emitted which undergoes a detectable change upon hybridization between a capture probe and its target sequence. Capture probes can be labeled such that they emit a signal in a target dependent manner by labeling a probe with a signal generating group (variably referred to in this embodiment as a “reporter group”) and a quenching group such that the signal generated by the reporter group is suppressed by the quenching group in the absence of the target sequence. Such reporter/quencher pairs have previously been described in U.S. Pat. No. 5,487,972 and U.S. Pat. No. 5,210,015 and may include, for example fluorophores such as rhodamine, coumarin, and fluorescein and well as derivatives thereof such as Tamra™ (6-carboxy-tetramethyl-rhodamine), Texas Red™, Lucifer Yellow, 7-hydroxy-coumarin, and 6-carboxy-fluorescein. Another example of a capture probe capable of generating a signal in a target dependent manner includes a probe labeled with a PORSCHA dye or an intercalating dye. PORSCHA dyes have been described in U.S. Pat. No. 5,332,659 and demonstrate a change in fluorescence based upon the proximity of one PORSCHA dye with another. Intercalating dyes have been described in PCT Application No. WO 95/01341, D. Figeys, et. al., Journal of Chromatography A, 669, pp. 205-216 (1994), and M. Ogur, et. al., BioTechniques 16(6) pp. 1032-1033 (1994); and demonstrate an increase in fluorescence intensity when associated with a double stranded nucleic acid sequence as opposed to the fluorescence intensity emitted by such a dye associated with a single stranded nucleic acid sequence.

Based upon the above discussion, those skilled in the art will recognize that the signal generating system can be broken down into component parts or “members of the signal generating system”. For example, a quenching group is one member of a reporter/quenching group signal generating system. Alternatively, for example, a single PORSHA dye is one member of a PORSHA dye signal generating system.

The unlabeled or labeled capture probes, as well as unlabeled or labeled primer sequences that can be employed according to the present invention, can be prepared by any suitable method. For example, chemical synthesis of oligonucleotides has previously been described in, for example, U.S. Pat. No. 4,458,066, U.S. Pat. No. 4,415,732 and U.S. Pat. No. 4,948,882.

A “defined pattern” of capture probes immobilized to the solid support material means that the sequence of a capture probe immobilized at a particular site on the support material is known. The pattern may be as simple as at least two different oligonucleotides spotted on a planar support material. More complex patterns, such as support materials having more than at least two sites having different capture probes immobilized thereon, can also be employed and have been described in U.S. Pat. No. 5,405,783, U.S. Pat. No. 5,412,087, Southern E. M., et. al., Nucleic Acids Research, Vol. 22, No. 8, pp. 1368-1373 (1994) and Maskos U., et. al., Nucleic Acids Research, Vol. 21, No. 20, pp. 4663-4669 (1993). In any case, the pattern is defined and therefore, the sequence of a capture probe or capture probes at a particular site on the support material is known.

Capture probes may be bound to a support material using any of the well known methodologies such as, for example, adsorption, covalent linkages, specific binding member interactions, or gold thiolate interactions. Capture probes also can be synthesized directly to the support material as described in U.S. Pat. No. 5,405,783, and U.S. Pat. No. 5,412,087.

After a test sample is contacted with the hybridization platform, the capture probes hybridize with their respective target sequences, if present, to thereby immobilize the target sequences to the hybridization platform. Upon hybridization with a target sequence, the signal generating groups associated with a capture probe produce a detectable change in signal. The change is generally dependent upon the signal generating system associated with the probe, and such a change may be detectable upon hybridization of the target sequence with the capture probe.

For example, in the case where a capture probe is labeled with an intercalation dye, the fluorescent signal emitted from the dye increases in intensity upon hybridization between the capture probe and its complementary target sequence. Prior to hybridization, the capture probe has a signal of a given intensity and when the capture probe is hybridized with the target sequence, the signal has a different intensity. This change in intensity can be detected as an indication that the target sequence is hybridized to the capture probe and therefore present in the test sample.

Alternatively, in the event a capture probe is labeled with a PORSCHA dye, a complementary target sequence labeled with another PORSCHA dye will change the spectral properties of the PORSCHA dye on the capture probe upon hybridization. The target sequence can be labeled with a PORSCHA dye before or after hybridization between the capture probe and target sequence by contacting the target sequence with a conjugate comprising a specific binding member conjugated to a PORSCHA dye. Specific binding members are well known and may include, for example, antibodies and antigens, haptens and antibodies, biotin and avidin, complementary nucleic acid sequences and the like. Alternatively, the target sequence can be amplified using an amplification primer labeled with a PORSCHA dye. Any of these methods can be employed to label a target sequence with a PORSCHA dye. Upon hybridization between a PORSCHA labeled target sequence and a PORSCHA labeled capture probe, the change in signal can be detected as an indication of the presence of the target sequence on the hybridization platform and therefore the presence of the target sequence in the test sample.

In one embodiment, hybridization between amplified products and immobilized capture nucleic acid sequences is carried out under the following hybridization conditions. An incubation of about an hour at 42° C. in a solution comprising 0.2 M sodium phosphate (pH 7.0), 7.1×TBS, 0.1% SDS and 0.08 N HCl, followed by room temperature washes in a solution comprising 0.1×SSC and 0.1% SDS). Hybridization can be carried out under conditions of higher or lower stringency, with a possible inclusion in the hybridization solution of any one of Denhardt's solution, sheared salmon sperm DNA, dextran sulfate and SSC. Changes in the stringency of hybridization and signal detection are primarily accomplished through the manipulation of formamide concentration (lower percentages of formamide result in lowered stringency); salt conditions, or temperature. For example, lower stringency conditions include an incubation at 37° C. in a solution comprising 6×SSPE (20×SSPE=3M NaCl; 0.2M NaH₂PO₄; 0.02M EDTA, pH 7.4), 0.5% SDS, 30% formamide, 100 μg/ml salmon sperm blocking DNA; followed by washes at 50° C. with 1×SSPE, 0.1% SDS. In addition, to achieve even lower stringency, washes performed following stringent hybridization can be done at higher salt concentrations (e.g. 5×SSC).

Note that variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

Internal Control

An internal control containing a synthetic fragment flanked by sequences amplifiable by the primers used in the assay is used to monitor sample recovery during extraction, amplification and detection. An internal control is a nucleic acid sequence, unrelated to any capture nucleic acid sequence used in the assay, flanked by sequences amplifiable by the primers used in the assay. In one embodiment, the following sequence was used:

5′ GAAAGCCCTACGAACCACTGAAAGTCCGAGATGTAGGGGGCTGTTGAA
AAAACCCTGGTGTGGGACAAGATACTCATCTGCATCCACAATGTCTTCCA
TGTCCTCCTCCTCTATCAGGGTGCCGATAAAACTTGGAATCTGTAGGGCT
AGGGCAAGTGCATCCTTTCATCTCCCTGTATAACAAGATAGCGGGGAGGG
TCACGAGCCATTTTGGAGAACTCTGCAATCAGCTCACGAAACTTGGGGCG
GCTGTCTGCATCACTCATCCAGCATTTGACCATGATCATGTACACATCAAT
GGTACAAATGGGTGGCTGGGGCAAACGCTCTCCCTTCTCCAAGACGGAGG
AGATTTCACTTGCGAGGTTGGTGAGTGATTGGAGGT 3′

where the underlined sequences are sequences that can hybridize to the disclosed HBV primers under the conditions employed in the amplification step.

### EXAMPLE 1

### Multiple Detection of HCV, HBV and HIV by PCR

Experimental

Nucleic Acid Isolation

Nucleic acids were extracted from human serum, plasma, or cultured viruses using the QIAmp spin column procedure (QIAGEN, CA). Purified nucleic acids were divided into aliquots and stored at −20° C. for later us.

Viral Fragment Amplification

Reverse transcription was carried out at 42° C. for30 minutes, 65° C. 5′ and 95° C. for 15 minutes with 40 units of M-MuLV RT in the presence of hexanucleotide mix, Uracil glycosylase (UNG) and 100 uM dNTP; PCR for viral fragments was carried out with biotinated oliogonucleotide primers targeted at HIV gag (HIV 1f, HIV2f, HIV 1r and HIV2r), HCV 5′utr (HCVf1 and HCVr 1) and HBV s-gene (HBV 1 f and HBV 1 r) simultaneously at 94° C. for 45 seconds; 55° C. for 45 seconds; 72° C. for 60 seconds for 35 to 45 cyclers, then 72° C. for 10 minutes. The final selected primers are listed below (x:biotin) HBV primers:

| | | |
|---|---|---|
| HBV primers: | 5′-xACCTCCAATCACTCACCAACCT-3′ | (22 bases); |
| | 5′-xGAAAGCCCTACGAACCACTGAA-3′ | (22 bases). |
| HCV primers: | 5′-xCCTATCAGGCAGTACCACAAGG-3′ | (22 bases);. |
| | 5′-xCGCTCTAGCCATGGCGTTAGTA-3′ | (22 bases). |
| HIV- 1 -M primers: | 5′xCTATTTGTTC(C/T)TGAAGGGTACTAGTA-3′ | (27 bases); |
| | 5′-ATACCCATGTT(C/T)(A/T)CAGCATTATCAGA-3′ | (26 bases). |
| HIV- 1 -O primers: | 5′-x(G/T)AATTTGCTCTTGCTG(G/T)GTGCTAGTT-3′ | (26 bases); |
| | 5′-xATTCCTATGTT(C/T)ATGGCATT(G/A)TCAGA-3′ | (26 bases). |

External Amplification Controls

Steps taken to monitor DNA extraction, amplification, and detection were as follows: (1) A positive plasma sample with known viral load was used as a positive control; (2) a negative amplification control was included, in which the RT-PCR reaction mixture contained nuclease-free water instead of purified nucleic acids; and (3) negative clinic control from a healthy donor was also included.

Internal Control For Viral Amplification

Two long, synthetic oligomers, with the corresponding primer sequence of HBVf1 and HBVr1 at the 5′ site, were synthesized to form 360 bp fragment as an unrelated internal control to monitor nucleic and extraction and subsequent amplification. The full-length internal control fragment was cloned into TA vector. Known amounts of purified internal control were added to plasma or serum samples prior to extraction to monitor nucleic acid yield. Since the full length internal control contains sequences complementary to PCR primers (HBVf1 and HBvr1), viral amplification can be monitored by co-amplyfing the internal control fragment

DNA Sequencing

PCR products were electrophoresized through a 1.5% agarose gel in 1×TBE buffer. DNA was excised from agarose gel and purified using the QIAcuick Gel Extraction Kit (Qiagen, Calif.). Cycle sequencing reaction was performed on an ABI Thermocycler 9600. Excess fluorescent dideoxy terminators were removed from the DNA sequencing reaction by centrifugation through Centri-Sep columns (Princeton Separations, N.J.). Reaction products were analyzed on 6% polyacrylamide/urea gel with an Applied Biosystem 373×1 DNA Sequencer. Viral sequences were aligned and phylogenetic trees were confirmed using the neighbor-joining method or BLAST-based analysis (GDB,NLM).

Preparation Of Microtiter Plates

Specific oligomer probe for HIV, HBV and HCV or mixes were attached to the plates by a carbodiimide-mediated condensation reaction resulting in a covalent attachment of the capture probes to the microtubes (Rasmussen, S. R., et al., Anal. Biochem. 198:138-142 (1991)). Specific immobilized oligomer on the plate captured biotin-labeled PCR products preferentially by DNA-DNA hybridization due to the presence of complementary sequence either to viruses or to the random sequence of the internal standard template.

Briefly, a freshly made 100 μl coating mix consisting of 100 nM capture oligomer and 10 mM EDC (1-ethyl-3-(3-dimethylaminopropyl)-carbodiimide) (Sigma) in 10 mM 1-methyl-imidazole (1-MeIm) (pH 7.0) was added to each well. A total of capture probes (5′-phosphorylated oligonucleotide) was about 10 pmol per well. NucleoLing Strips were incubated at 50° C. for 4-24 hours, and wells were washed three times with freshly prepared and pre-warmed 0.4 M NaOH and 0.25% Tween 20, pre-warmed to 50° C. Residual NaOH was removed by extensive washing with distilled water at Room Temperature and dried for use.

Capture probes HIV-cap1 and HIV-cap2 were designed for detecting HIV-1 subtype M and FIV subtype O, respectively. Capture probe HBV-cap1 was designed for detecting HBV subtype a to f, and capture probe HCV-cap1 was designed for detecting all of the subtypes of HCV. A mix of HIV-cap1, HIV-cap2, HBV-cap1 and HCV-cap1 in the same microtiter wells was used for screening any bloodborne viruses. The capture probe for internal control, Viralcap-IC, was used to detect internal control fragment forcalibrating the assay.

The following protocol was used to make plates for HBV, HIV-1 type M and multiplates per 5 plates:

plate= Nucelolink strips # 248259
units per sleeve/case= 12/120
pmol/well=10

| | |
|---|---|
| | 10 pmol/well × 100 × 5 = 5000 pmol |
| oligo = (1) | HIV-1-0-CAP-L 413.3 pmol/μl |
| | 5000 ∓ 413.3 pmol/μl = 12.1 μl |
| (2) | HBV-CAP-30-1 92.4 pmol/ul |
| | 5000 ∓ 92.4 pmol/ul = 54.1 μl |
| (3) | HCV-CAP-R 198.8 pmol/ul |
| | 5000 ∓ 198.8 pmol/ul = 25.2 μl. |
| (4) | HIV-1-all-CAP-L GIBCOBRL |
| | 24.3 nmol dissolve with 200 distilled water |
| final conc = | 24300 pmol/200 μl |
| = | 121.5 pmol/ul |
| 5000 ∓ 121.5 pmol/ul = | 41.2 μl |
| 50 ml 10 mM MeIm. | |
| | 5 plates |
| 100 mg EDC | |

Detection Of Amplification Products By Capture Hybridization Assay

Biotin-labeled amplified PCR product was added to the Nucleolink tube and denatured with NaOH. PCR products were hybridized to-the covalently linked probe on the microtiter plate and detected with streptavidin-peroxidase conjugate calorimetrically. The optical density at 45ₙₘ was recorded in files using a PC driven plate reader and the negative controls in the assay were used to set up the cutoff level for positive samples. The following protocol was used for the capture assay:

1. Add 10 μl of the PCR product to the Nucleolink wells with the solid phase capture oligomer covalently bound.

2. Add 10 μl of 1N NaOH and mix well with pipet tips.

3. Incubate for 10 min. at RT.

4. To each well, add 100 μl of hybridization buffer and mix well with pipet tips Hybridization buffer: 50 ml mixwell

| | |
|---|---|
| 1M phosphate PH 7.0 | 10 ml |
| 1OX TBS | 35.7 ml |
| 10% SDS | 0.5 ml |
| 1N HCL | 3.8 ml |

Before use the hybridization buffer prewarm at 42° C. water bath.

5. Incubate for 1 hour at 37-42° C. and seal tightly with tape.

6. Empty wells by vigorously shaking out the liquid, and wash wells with 200 μl of wash buffer 1 (0.1×SSC and 0.1% SDS) at room temperature for 3 min by shaking in an orbital shaker. Repeat 4 times.

7. Add 200 μl of blocking solution into each well and incubate at room temperature for 10 minutes in an orbital shaker.

8. Empty wells and add 100 μl of working conjugate solution. Shake for 10 min on an orbital shaker at room temperature.

9. Wash wells with 200 μl of Wash Buffer 2 for 3 min twice.

Wash Buffer 2:

| | |
|---|---|
| Glycerol | 125 ml |
| 10% SDS | 5 ml |
| 10x TBS | 50 ml |

add dH20 to 500 ml.

10. Wash wells with 200 μl of Wash Buffer 3 for 3 min twice.

Wash buffer 3:

| | |
|---|---|
| Glycerol | 125 ml |
| 10x TBS | 50 ml |

add dH20 to 500 ml.

11. Add 100 μl of working substrate solution (TBM system) and develop in the dark for 30 minutes.

12. Stop the reaction by adding 100 μl of 2N Sulfuric Acid, mix well and read absorbance of the wells at 450 nm within 30 min of adding the stop solution.

Summary

A universal amplification and detection procedure was developed to screen retrovirus (HIV), RNA virus (HCV) and DNA virus (HBV) simultaneously. Degenerate primers were designed to ensure that amplification of all subtypes of HIV-1-M, HIV-1-O, HCV and HBV. Viral fragments were PCR-amplified with biotin labeled primers after reverse transcription with random hexanucleotides. The biotin labeled PCR products were then hybridized to capture plates in which viral-specific or internal control oligonucleotide capture probes were immobilized on 96-well microplate through covalent attachment of phosphate-modified oligomer capture sequences to micro-plate strips. The presence of bloodborne viral sequences of HCV, HBV and HIV was determined by a microplate reader with a colorimetric reaction using streptavidin conjugated alkaline phosphatase and substrate.

Discussion

Non-discriminative amplification among the following viral subtypes has been verified:

HBV: A, B, C and D

HCV: 1, 2, 3, 4

HIV-1-M: A, B, C, D, E, F and

HIV-1-O

Extensive controls with characterized samples have been tested, incuding:

internal control and dUTP/Uracil Glycosylase;

sero-conversion panels and run controls; and

worldwide viral subtype collections.

The multiplexed screening of the present invention is capable of detecting HBV; HCV; HIV-1-M; and HIV-1 type O simultaneously. Three copies per assay, equivalent of 100 copies per mL are detected consistently without the requirement for a virus precentrifugation step. All major subtypes of HBV, HCV and HIV-1 including HIV-1 type O have been confirmed.

Results from the assay are summarized in tables 2, 3 and 4.

TABLE 2
HBV Panel: PHM935

| Bleed | | | |
|---|---|---|---|
| (Days) | Roche | HBV | HBV-IC |
| 2 | <400 | Negative | Positive |
| 7 | <400 | Negative | Positive |
| 9 | 600 | Positive | Positive |
| 14 | 800 | Positive | Positive |
| 16 | 500 | Positive | Positive |
| 21 | 9000 | Positive | Positive |
| 23 | 8000 | Positive | Positive |
| 28 | 80000 | Positive | Positive |
| 30 | 100000 | Positive | Positive |
| 35 | 400000 | Positive | Positive |
| 50 | 20000000 | Positive | Positive |
| 66 | 5000000 | Positive | Positive |
| 68 | 40000000 | Positive | Positive |
| 85 | 40000000 | Positive | Positive |
| 93 | 30000000 | Positive | Positive |
| 100 | 2000000 | Positive | Positive |
| 107 | 90000 | Positive | Positive |
| 114 | 30000 | Positive | Positive |
| 121 | 20000 | Positive | Positive |
| 123 | 7000 | Positive | Positive |
| 128 | 4000 | Positive | Positive |
| 135 | 1000 | Positive | Positive |
| 144 | <400 | Positive | Positive |
| 151 | 500 | Positive | Positive |
| 158 | 700 | Positive | Positive |
| 165 | 800 | Positive | Positive |
| 170 | 900 | Positive | Positive |
| 175 | 2000 | Positive | Positive |
| 182 | 600 | Positive | Positive |
| 189 | 800 | Positive | Positive |
| 196 | <400 | Positive | Positive |
| 203 | <400 | Positive | Positive |

TABLE 3
HCV Genotypes and Titers

| BBI-ID | Genotypes | Copies/ml |
|---|---|---|
| E6-0508-0162 | 1a | 7 × 10⁴ |
| BZ6-1511-0013 | 1b | 3 × 10⁵ |
| E8-1702-0197 | 1b | 3 × 10⁶ |
| JE6-3107-0005 | 2a | 4 × 10³ |
| JE6-3107-0008 | 2a | 2 × 10⁴ |
| E8-1702-0254 | 2b | 1 × 10⁵ |
| E8-1404-0087 | 3a | 4 × 10⁵ |
| CT8-1509-00004 | 4a | 1 × 10⁴ |
| CT8-1509-0003 | 4a | 7 × 10⁴ |
| KG-2808-0030 | 6b | 9 × 10³ |

TABLE 4
Capture Assay for Normal Plasma

| | Ave | StaDev | Cut-off* |
|---|---|---|---|
| HBV | 0.06 | 0.06 | 0.24 |
| HCV | 0.05 | 0.05 | 0.20 |
| HIV | 0.05 | 0.04 | 0.25 |
| IC | 0.52 | 0.20 | |

*Cut-off OD 450 nm for 24 Negative Samples tested is calculated as the average OD plus three times of StaDev. 3 copies of Internal Control (IC) per assay were spiked.

### Example 2

### Multiplex Detection of HIV, HCV and HBV Using TMA or NASBA

The currently developed multiplex assay for HIV, HCV and HBV can be carried out using other amplification-based assay in addition to PCR, including transcription mediated TMA or NASBA, ligation based amplification and others. A detailed example for Transcription-mediated amplification multiplex assay is described below.

Attaching a T7 promoter sequence (5′-biotinylated-AAT TTA ATA CGA CTC ACT ATA GGG) at the 5′ site of any specific viral PCR primers (HIV, HCV and HBV) described above, enables transcription mediated amplification of nucleic acids of HIV, HCV and HBV.

Capture probes for HIV, HCV and HBV, as well as the internal control, can be the same as for the PCR-based assay. In one aspect of the invention biotin is located in each of primers, allowing transcription-mediated, amplified products to be detected with colorimetric reaction after hybridization with the capture probe on plates.

The transcription mediated amplification can be a two enzyme system (Reverse transcriptase from AMV, MMLV, HIV or modified RT, plus T7 RNA polymerase), or a three enzyme system (Reverse transcriptase from AMV, MMLV, HIV or modified RT, T7 RNA polymerase, plus Rnase H). For example, three enzyme reaction can be conducted at 37° C. for 30 to 90 minutes in 50 to 200 μl containing 60 mM Tris HCl (pH 8.2), 10 mM MgCl₂, 10 mM KCl, 2 mM spermidine-HCl, 2.5 mM dithiothreitol, 0.5 mM of each the dATP, dTTP, dCTP and dGTP, 2 mM each of ATP, UTP, CTP and GTP, 20 pmol each biotinated T7 attached primers (HIV, HCV and HBV), nucleic acid extraction from human plasma as amplification template. 90 μg HIV-1 RT, 100 μg of T7 RNA polymerase, and 2 units of E. coli RNAse H.

If viral PCR primers of HIV, HCV and HI3V are attached at 5′ site with T3 promoter sequence, rather than T7 promoter, transcription-mediated amplification can be performed when T3 RNA polymerase replaces T7 RNA polymerase in the TMA or NASBA reaction mix.

Although the foregoing refers to particular preferred embodiments, it will be understood that the present invention is not so limited. It will occur to those of ordinary skill in the art that various modifications may be made to the disclosed embodiments and that such modifications are intended to be within the scope of the present invention, which is defined by the following claims.

All publications, patents and patent applications mentioned in this specification are indicative of the level of skill of those in the art to which the invention pertains. All publications, patents and patent applications are herein incorporated by reference to the same extent as if each individual publication or patent application was specifically and individually indicated to be incorporated by reference in their entirety.

What is claimed is:

1. A method for detecting the presence of multiple viral agents in a test sample, comprising:
a. carrying out an amplification reaction amplifying nucleic acids from at least one or more of HIV, HCV and HBV using a mixture of primers specific for HBV, HCV, HIV-1 type M and HIV-1 type O; and
b. detecting for the presence of amplified nucleic acids and determining whether said nucleic acids are associated with at least HIV, HCV, or combinations thereof.

2. The method of claim 1, wherein an internal control amplifiable by any said primers is added to said sample prior to step (a).

3. The method of claim 2 wherein said internal control is a nucleic acid amplifiable by labeled primers specific for one of HBV, HCV or HIV.

4. The method of claim 1 wherein said sample is a bodily fluid or tissue.

5. The method of claim 1 where said sample is selected from the group consisting of whole blood, plasma, serum or white blood cells.

6. The method of claim 1 wherein said nucleic acids are amplified by PCR.

7. The method of claim 1 wherein said primers specific for HBV are selected from the group consisting of
a. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 300 to nucleotide 400, or a portion thereof comprising at least the sequence 340 to 350; and
b. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 650 to nucleotide 750, or a portion thereof comprising at least the sequence 710 to 720.

8. The method of claim 1 wherein said primers specific for HCV are selected from the group consisting of
a. polynucleotides capable of hybridizing under stringent conditions to a sequence of HCV polyprotein gene (GenBank accession number (A#) AF271632) or the complement thereof, wherein said sequence is from nucleotide 50 to nucleotide 150, or a portion thereof comprising at least the sequence 83 to 93; and
b. polynucleotides capable of hybridizing under stringent conditions to a sequence of HCV strain MD 12 complete genome (GenBank accession number (A#) AF207753) or the complement thereof, wherein said sequence is from nucleotide 220 to nucleotide 320, or a portion thereof comprising at least the sequence 271 to 281.

9. The method of claim 1 wherein said primers specific for HIV are selected from the group consisting of polynucleotides of 10 to 100 bases capable of hybridizing under stringent conditions to a nucleic acid having a sequence selected from the group consisting of
a. 5′ ATACCCATGTT(C/T)(A/T)CAGCATTATCAGA 3′;
b. 5′ CTATTTGTTC(C/T)TGAAGGGTACTAGTA 3′;
C. 5′ (G/T)AATTTGCTCTTGCTG(G/T)GTGCTAGTT 3′;
d. 5′ ATTCCTATGTT(C/T)ATGGCATT(G/A)TCAGA 3′; or
e. a complement of any one of the sequences listed in a to d.

10. The method of claim 1 wherein said nucleic acids are amplified by TMA.

11. The method of claim 1 wherein said nucleic acids are amplified by NASBA.

12. The method of claim 10 or 11 wherein said primers specific for HBV are selected from the group consisting of
a. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 300 to nucleotide 400, or a portion thereof comprising at least the sequence 340 to 350; and
b. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 650 to nucleotide 750, or a portion thereof comprising at least the sequence 710 to 720;
Wherein at least one of a or b further includes a T7 or T3 promoter sequence.

13. The method of claim 10 or 11 wherein said primers specific for HCV are selected from the group consisting of
a. polynucleotides capable of hybridizing under stringent conditions to a sequence of HCV polyprotein gene (GenBank accession number (A#) AF271632) or the complement thereof, wherein said sequence is from nucleotide 50 to nucleotide 150, or a portion thereof comprising at least the sequence 83 to 93; and
b. polynucleotides capable of hybridizing under stringent conditions to a sequence of HCV strain MD12 complete genome (GenBank accession number (A#) AF207753) or the complement thereof, wherein said sequence is from nucleotide 220 to nucleotide 320, or a portion thereof comprising at least the sequence 271 to 281.
Wherein at least one of a or b further includes a T7 or T3 promoter sequence.

14. The method of claim 10 or 11 wherein said primers specific for HIV are selected from the group consisting of polynucleotides of 10 to 100 bases capable of hybridizing under stringent conditions to a nucleic acid having a sequence selected from the group consisting of
a. 5′ ATACCCATGTT(C/T)(A/T)CAGCATTATCAGA 3′;
b. 5′ CTATTTGTTC(C/T)TGAAGGGTACTAGTA 3′;
c. 5′ (G/T)AATTTGCTCTTGCTG(G/T)GTGCTAGTT 3′;
d. 5′ ATTCCTATGTT(C/T)ATGGCATT(G/A)TCAGA 3′; or
e. a compliment of any one of the sequences listed in a to d;
Wherein at least one of a through e further includes a T7 or T3 promoter sequence.

15. The method of any of claims 12 to 14 wherein said T7 or T3 promoter sequence is at the 5′ end of said primers.

16. The method according to claim 1 wherein said primers are labeled with biotin.

17. The method according to claim 1 wherein said primers are labeled with a fluorophore.

18. The method according to claim 1 wherein said primers are labeled with a radioactive isotope.

19. The method according to claim 1, wherein prior to step a, viral nucleic acids are extracted from said sample is a single extraction step.

20. The method according to claim 1, wherein after step a, any amplified products are captured on a plurality of microtiter wells by hybridization to an immobilized capture nucleic acid, wherein each microtiter well includes an immobilized capture nucleic acid specific for one of HIV, HCV and HBV.

21. A kit for the detection of HIV, HCV, HBV and combinations thereof in blood or a blood product sample comprising:
a. primers specific for HBV;
b. primers specific for HCV;
c. degenerate primers specific for HIV-1 type M;
d. primers specific for HIV-1 type 0;
e. capture nucleic acid specific for HVB;
f. degenerate capture nucleic acid specific for HCV;
g. degenerate capture nucleic acid specific for HIV-1 type M; and
h. capture nucleic acid specific for HIV-1 type O.

22. The kit of claim 21 further comprising a plurality of wells, wherein at least one well contains an immobilized capture nucleic acid specific for HIV, at least one well contains an immobilized capture nucleic acid specific for HCV, at least one well contains an immobilized capture nucleic acid specific for HBV.

23. The kit of claim 21 further comprising at least one well containing immobilized capture nucleic acid specific for the internal control nucleic acid.

24. The kit of claim 21 further comprising at least one empty well.

25. The kit of claim 21 wherein said wells are arranged in a microtiter plate.

26. The kit of claim 21 wherein said primers specific to HIV are selected from the group consisting of polynucleotides of 10 to 100 bases capable of hybridizing under stringent conditions to a nucleic acid having a sequence selected from the group consisting of
a. 5′ ATACCCATGTT(C/T)(A/T)CAGCATTATCAGA 3′;
b. 5′ CTATTTGTTC(C/T)TGAAGGGTACTAGTA 3′;
C. 5′ (G/T)AATTTGCTCTTGCTG(G/T)GTGCTAGTT 3′;
d. 5′ ATTCCTATGTT(C/T)ATGGCATT(G/A)TCAGA 3′; or
e. a complement of any one of the sequences listed in a to d.

27. The kit of claim 21 wherein said primers specific to HBV are selected from the group consisting of
a. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 300 to nucleotide 400, or a portion thereof comprising at least the sequence 340 to 350; and
b. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 650 to nucleotide 750, or a portion thereof comprising at least the sequence 710 to 720.

28. The kit of claim 21 wherein said primers specific to HCV are selected from the group consisting of
a. polynucleotides capable of hybridizing under stringent conditions to a sequence of HCV polyprotein gene (GenBank accession number (A#) AF271632) or the complement thereof, wherein said sequence is from nucleotide 50 to nucleotide 150, or a portion thereof comprising at least the sequence 83 to 93; and
b. polynucleotides capable of hybridizing under stringent conditions to a sequence of HCV strain MD12 complete genome (GenBank accession number (A#) AF207753) or the complement thereof, wherein said sequence is from nucleotide 220 to nucleotide 320, or a portion thereof comprising at least the sequence 271 to 281.

29. A kit of claim 21 wherein said capture sequence specific to HBV include polynucleotides of 10 to 100 bases capable of hybridizing under stringent conditions to a nucleic acid having the sequence:
5′ACTAGTAAACTGAGCCAGGAGAAACGGACT3′
or the complement thereof.

30. A kit of claim 21 wherein said capture sequence specific to HCV include polynucleotides of 10 to 100 bases capable of hybridizing under stringent conditions to a nucleic acid having the sequence:
5′CTAGCCGAGTAG(C/T)GTTGGGT(C/T)GCG 3′
or the complement thereof.

31. A kit of claim 21 wherein said capture sequence specific to HIV-1 type M include polynucleotides of 10 to 100 bases capable of hybridizing under stringent conditions to a nucleic acid having the sequence:
5′AATGAGGAAGCTGCAGAATGGGAYAG 3′
or the complement thereof.

32. A kit of claim 21 wherein said capture sequence specific to HIV-1 type O include polynucleotides of 10 to 100 bases capable of hybridizing under stringent conditions to a nucleic acid having the sequence:
5′ AAGGAAGTAATCAATGAGGAAGCAG 3′
or the complement thereof.

33. The kit of claim 21 wherein said primers further comprise a T7 or T3 promoter sequence.

34. A kit comprising one or more vials containing a primer specific to HIV, HBV, HCV or combinations thereof; wherein said primers specific to HIV are selected from the group consisting of polynucleotides of 10 to 100 bases capable of hybridizing under stringent conditions to a nucleic acid having a sequence selected from the group consisting of
a 5′ ATACCCATGTT(C/T)(A/T)CAGCATTATCAGA3′;
b. 5′ CTATTTGTTC(C/T)TGAAGGGTACTAGTA 3′;
C. 5′(G/T)AATTTGCTCTTGCTG(G/T)GTGCTAGTT 3′;
d. 5′ ATTCCTATGTT(C/T)ATGGCATT(G/A)TCAGA 3′; or
e. a complement of any one of the sequences listed in a to d;
wherein said primers specific to HBV are selected from the group consisting of
f. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 300 to nucleotide 400, or a portion thereof comprising at least the sequence 340 to 350; and
g. polynucleotides capable of hybridizing under stringent conditions to a sequence of HBV strain 1366h pre-S2-S protein (S) gene (GenBank accession number (A#) AF214659) or the complement thereof, wherein said sequence is from nucleotide 650 to nucleotide 750, or a portion thereof comprising at least the sequence 710 to 720; and
wherein said primers specific to HCV are selected from the group consisting of
h. polynucleotides capable of hybridizing under stringent conditions to a sequence of HCV polyprotein gene (GenBank accession number (A#) AF271632) or the complement thereof, wherein said sequence is from nucleotide 50 to nucleotide 150, or a portion thereof comprising at least the sequence 83 to 93; and
i. polynucleotides capable of hybridizing under stringent conditions to a sequence of HCV strain MD12 complete genome (GenBank accession number (A#) AF207753) or the complement thereof, wherein said sequence is from nucleotide 220 to nucleotide 320, or a portion thereof comprising at least the sequence 271 to 281.

35. The kit of claim 21 or 34 wherein said primers are labeled with biotin.

36. The method of claim 21 or 34 wherein said primers are labeled with a fluorophore.

37. The method of claim 21 or 34 wherein said primers are labeled with a radioactive isotope.

38. The kit of claim 21 or 34 wherein said primers are lyophilized.

39. The kit of claim 21 or 34 wherein said primers are in liquid form.

40. A kit comprising capture nucleic acids specific to HBV, HCV, HIV-1 type M, HIV-1 type O or combinations thereof linked to solid support.

41. A kit of claim 41 wherein said solid support is a bead.

42. A kit of claim 41 wherein said solid support is a well.

43. A kit of claim 41 wherein said solid support is a vial.

44. A kit of claim 41 wherein said solid support is a membrane.

45. A kit of claim 41 wherein said solid support is a tube.

46. A kit of claim 41 wherein said solid support is a capillary tube.
